(19) United States
(12) Patent Application Publication
(10) Pub. No.: US 2007/0232924 A1
(43) Pub. Date: Oct. 4, 2007

(54) Ultrasonic probe and ultrasonic diagnosing apparatus

(75) Inventor: Hiroyuki Karasawa, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/717,102

(22) Filed: Mar. 13, 2007

(30) Foreign Application Priority Data
Mar. 13, 2006 (JP) 2006-067731

(51) Int. Cl. A61B 8/14

(52) U.S. Cl. 600/459

(57) ABSTRACT

N numbers of ultrasonic transducers are arranged at a tip of an ultrasonic probe. A wire from each transducer is connected to a multiplexer (MUX). The MUX selectively switches M numbers of the ultrasonic transducers to be driven out of the N numbers of the ultrasonic transducers. A short-circuit switch is connected to each of the ultrasonic transducers. The short-circuit switches short-circuit adjacent L numbers of the ultrasonic transducers, and simultaneously drive the L numbers of the ultrasonic transducers as a single ultrasonic transducer. A CPU changes the number L in accordance with the frequency band of the ultrasonic waves which is set by an operation unit.

### BACKGROUND OF THE INVENTION

1. Field of the Invention

The present invention relates to an ultrasonic probe having a plurality of ultrasonic transducers on a tip thereof, and an ultrasonic diagnosing apparatus having the ultrasonic probe and a processor for generating an ultrasonic image.

2. Description Related to the Prior Art

Recently, ultrasonic images are widely used for medical diagnoses in the medical field. The ultrasonic image is obtained by emitting ultrasonic waves from an ultrasonic probe toward a body part of a patient, and electrically processing echo signals from the body part by a processor.

It is also possible to obtain an ultrasonic tomographic image by scanning the body part with the ultrasonic waves. The ultrasonic probes of a mechanical scan type and an electronic scan type are widely recognized. The mechanical scan type probes mechanically rotate, swing, or slide ultrasonic transducers. The electronic scan type probe has plural ultrasonic transducers arranged in an array, and selectively switches the ultrasonic transducers to be driven using an electronic switch or the like.

The electronic scan type probes include convex scan type probes and radial scan type probes. The convex scan type probe has plural ultrasonic transducers arranged in a sector shape on a tip of the probe. The radial scan type probe has plural ultrasonic transducers arranged on an outer periphery of a cylindrical tip of the probe.

The convex scan type probe or the radial scan type probe can be inserted into a forceps opening of an electronic endoscope, or combined with an imaging device to be a so-called ultrasonic endoscope, which is inserted into a body cavity for intracavity diagnosis. To reduce the physical burdens of the patients, various attempts have been made to reduce diameters of the ultrasonic probes.

For instance, Japanese Patent Laid-Open Publication No. 2005-034634 suggests an ultrasonic diagnosing apparatus having a 2D vibrator array (which corresponds to the ultrasonic transducer) constituted of plural sub arrays. Each sub array is divided into plural groups. Ultrasonic waves and echo signals are transmitted and received on the group basis by multiplexers, and thus the number of connecting wires between the ultrasonic probe and the processor is reduced, which in result reduces the diameter of the ultrasonic probe.

In order to obtain accurate ultrasonic tomographic images for a medical diagnosis, it is necessary to adjust the frequency of the ultrasonic waves in accordance with a depth of a body part of interest, because there are individual differences (for instance, a fat amount) among patients, and different propagation characteristics among tissues. For instance, high-frequency ultrasonic waves can provide high resolution images, but cannot provide clear images of deeper regions. Therefore, it is likely that low-frequency ultrasonic waves are emitted at first to observe the whole body part of the interest including the deeper regions, and then high-frequency ultrasonic waves are emitted to minutely observe the body part which is suspected to be pathologic tissues.

For this reason, conventionally, plural ultrasonic probes having ultrasonic transducers of different frequency bands have been used in accordance with the body part of interest and/or the purpose of the diagnosis. However, insertion and removal of plural ultrasonic probes increase the physical burdens of the patient.

To solve the above problem, Japanese Patent Laid-Open Publication No. 05-056980 suggests an ultrasonic diagnosing apparatus using dual frequency transducers to obtain ultrasonic images in two frequency bands. In addition, Japanese Patent Laid-Open Publication No. 2004-222827 discloses an ultrasonic diagnosing apparatus using a sparse two-dimensional vibrator array (which corresponds to the ultrasonic transducer) having two types of receiving elements which receive fundamental waves and harmonic waves respectively. Thus, the ultrasonic diagnosing apparatus enables to use ultrasonic waves of a wide frequency band. In this ultrasonic diagnosing apparatus, receiving elements of the same type are short-circuited to reduce the number of connecting wires between an ultrasonic probe and a processor.

To carry out the ultrasonographic scanning over a wide frequency band by simultaneously driving a part of the ultrasonic transducers (an element group) and sequentially switching the element groups to be driven of the ultrasonic probe having plural ultrasonic transducers arranged, for instance, in a two-dimensional ultrasonic transducer array, it is necessary to provide different intervals between the ultrasonic transducers in accordance with the frequencies. To emit high frequency ultrasonic waves, it is necessary to reduce the interval between the ultrasonic transducers to form an appropriate ultrasonic beam without substantial side lobes. On the other hand, to emit low frequency ultrasonic waves, it is necessary to increase the width of the beam compared to that of the high frequency ultrasonic waves because a long focal distance is needed to focus such a low frequency ultrasonic beam. As a result, it is necessary to increase the number M of the ultrasonic transducers constituting the element group.

Since the number M of the ultrasonic transducers increases when the interval is reduced for the high frequency ultrasonic waves, the reduction of the connecting wires does not contribute significantly to reduce the diameter of the ultrasonic probe even if the multiplexers are used. On the other hand, if the interval is increased so as to obtain a certain beam width of the low frequency ultrasonic waves, the increased interval becomes inappropriate for the high frequency ultrasonic waves as described above.

The Japanese Patent Laid-Open Publication No. 2004-222827 does not disclose a solution to solve the above problems in using the ultrasonic waves of a wide frequency band, and could not achieve both the appropriate beam width for the ultrasonic waves of a wide frequency band, and the reduction of the probe diameter at the same time.

### SUMMARY OF THE INVENTION

A main object of the present invention is to provide an ultrasonic probe and an ultrasonic diagnosing apparatus capable of using ultrasonic waves of a wide frequency band with an appropriate beam width.

Another object of the present invention is to provide an ultrasonic probe and an ultrasonic diagnosing apparatus in which the number of wires is reduced to reduce a diameter of the ultrasonic probe.

To achieve the above and other objects, an ultrasonic probe according to the present invention includes N numbers of ultrasonic transducers disposed on a tip of the ultrasonic probe, a multiplexer for selectively switching M numbers of the ultrasonic transducers to be driven out of the N numbers of the ultrasonic transducers, and a short-circuit device for short-circuiting adjacent L numbers of the ultrasonic transducers out of the N numbers of the ultrasonic transducers to simultaneously drive the L numbers of ultrasonic transducers.

It is preferable that the number L is changed in accordance with a frequency band of the ultrasonic waves. It is preferable that the number L is changed in accordance with a focal distance of the ultrasonic waves. It is preferable that the number L is a submultiple of the number M.

It is preferable that the ultrasonic probe is inserted in a body cavity for intracavity diagnosis. Further, it is preferable that the ultrasonic probe is an ultrasonic endoscope having an image sensor for capturing an optical image of a target region.

It is preferable that the ultrasonic transducers transmit and receive the ultrasonic waves in a frequency band of approximately 3 to 20 MHz.

It is preferable that M numbers of the ultrasonic transducers are sequentially driven at a predetermined delay time.

An ultrasonic diagnosing apparatus is constituted of an ultrasonic probe having N numbers of ultrasonic transducers on a tip thereof, and a processor for generating an ultrasonic image from an echo signal from a target region received by the ultrasonic transducers. The ultrasonic probe includes a multiplexer for selectively switching M numbers of the ultrasonic transducers to be driven out of the N numbers of the ultrasonic transducers, and a short-circuit device for short-circuiting adjacent L numbers of the ultrasonic transducers out of the N numbers of the ultrasonic transducers to simultaneously drive the L numbers of the ultrasonic transducers. The processor includes a control unit for controlling an operation of the short-circuit device.

It is preferable that the ultrasonic diagnosing apparatus includes a first operation device for changing frequency band of the ultrasonic waves. It is preferable that the control unit changes the number L in accordance with an operation of the first operation device.

It is preferable that the ultrasonic diagnosing apparatus includes a second operation device for changing a focal distance of the ultrasonic waves. It is preferable that the control unit changes the number L in accordance with an operation of the second operation device.

It is preferable that the number L is a submultiple of the number M.

It is preferable that the ultrasonic probe is inserted into a body cavity for intracavity diagnosis.

It is preferable that the ultrasonic probe is an ultrasonic endoscope having an image sensor for capturing an optical image of a target region.

It is preferable that the ultrasonic transducers transmit and receive the ultrasonic waves in a frequency band of approximately 3 to 20 MHz.

It is preferable that the ultrasonic diagnosing apparatus further includes a driving device for sequentially driving the M numbers of the ultrasonic transducers at a predetermined delay time.

According to the ultrasonic probe and the ultrasonic diagnosing apparatus of the present invention, since the M numbers of the ultrasonic transducers to be driven are selectively switched among the N numbers of the ultrasonic transducers, and adjacent L numbers of the ultrasonic transducers are short-circuited to be simultaneously driven to change the width of the ultrasonic beam, the ultrasonic probe and the ultrasonic diagnosing apparatus are able to handle the ultrasonic waves of a wide frequency band while reducing the diameter of the ultrasonic probe.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a schematic diagram of an ultrasonic diagnosing apparatus; and

FIG. 2A, 2B, 2C, and 2D are explanatory views illustrating operation of short-circuit switches, with ultrasonic transducers driven at: 3.3 MHz in FIG. 2A, 5 MHz in FIG. 2B, 6.7 MHz in FIG. 2C, and 10 MHz in FIG. 2D.

### DESCRIPTION OF THE PREFERRED EMBODIMENTS

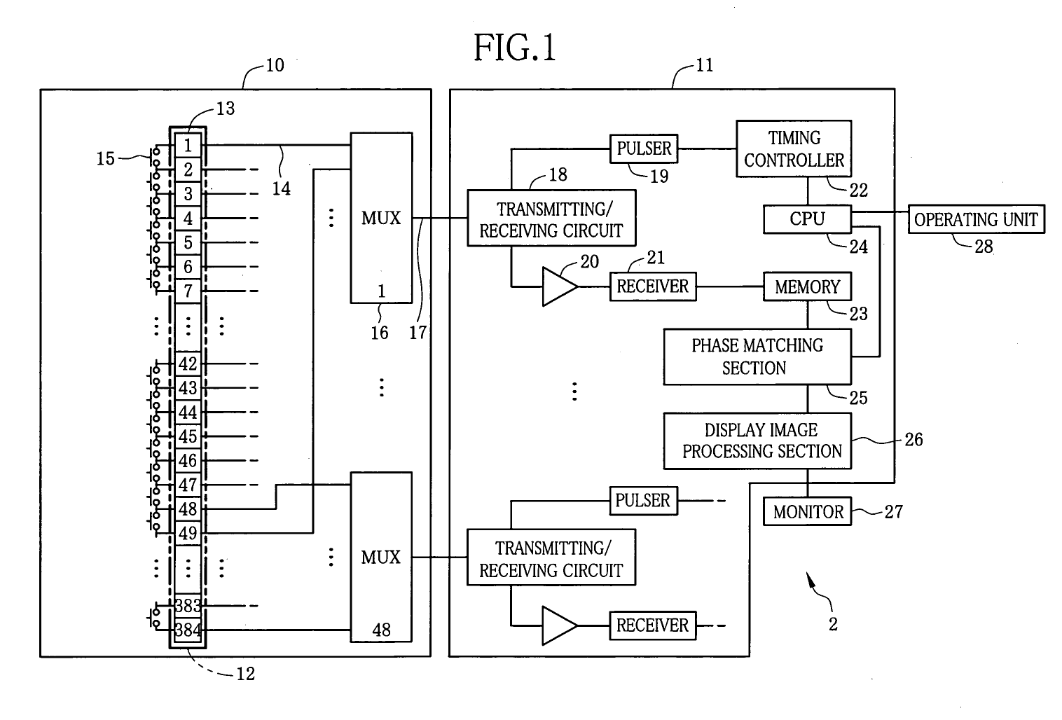

In FIG. 1, an ultrasonic diagnosing apparatus 2 is constituted of an ultrasonic probe 10 and a processor 11. The ultrasonic probe 10 is a fine probe with an outer diameter of, for instance, 10 mm to be inserted in a forceps opening of an electronic endoscope, or a so-called ultrasonic endoscope in which the probe and the electronic endoscope are integrated. The ultrasonic probe 10 is inserted into a body cavity of a patient.

On a tip the ultrasonic probe 10, an ultrasonic transducer array 12 is disposed. The ultrasonic transducer array 12 is, for instance, a radial scan type having N=384 ultrasonic transducers 13 arranged at 0.08 mm intervals on an outer periphery of a cylindrical support. For illustrative purpose, the ultrasonic transducers 13 are numbered from 1 to 384 in the drawing. It is also possible to use the ultrasonic transducer array 12 of a convex scan type having N numbers of ultrasonic transducers 13 arranged in a one-dimensional, one-and-half-dimensional, or two-dimensional array on a convex support.

The ultrasonic transducer 13 transmits and receives the ultrasonic waves of approximately 3 MHz to 20 MHz. The ultrasonic diagnosing apparatus 2 is capable of changing over the frequency bands in 5 levels, for instance, 3.3 MHz, 5 MHz, 6.7 MHz, 10 MHz, and 20 MHz. Thereby, an appropriate frequency is used for the diagnosis in accordance with purposes and applications. Specifically, to observe the entire body part including deeper regions, low frequency ultrasonic waves such as 3.3 MHz and 5 MHz are used. To observe a body part which is suspected to be pathologic tissues, high frequency ultrasonic waves such as 10 MHz and 20 MHz are used.

To the ultrasonic transducer 13, a wire 14 and a short-circuit switch 15 are connected. The wire 14 transmits excitation pulses output from a pulser 19 which will be described later, and echo signals received by the ultrasonic transducer 13.

The short-circuit switch 15 short-circuits adjacent ultrasonic transducers 13. To the short-circuit switch 15, a control line (not shown) from a CPU 24 is connected. The CPU 24 will be described later. An operation of the short-circuit switch 15 is controlled by control signals input from the CPU 24 via the control line.

To drive the ultrasonic transducers 13 at the frequency band of 20 MHz, all the short-circuit switches 15 are turned off, and each ultrasonic transducer is individually driven. On the other hand, when the short-circuit switches 15 are turned on, adjacent L numbers of ultrasonic transducers 13 connected by the short-circuit switches 15 are simultaneously driven.

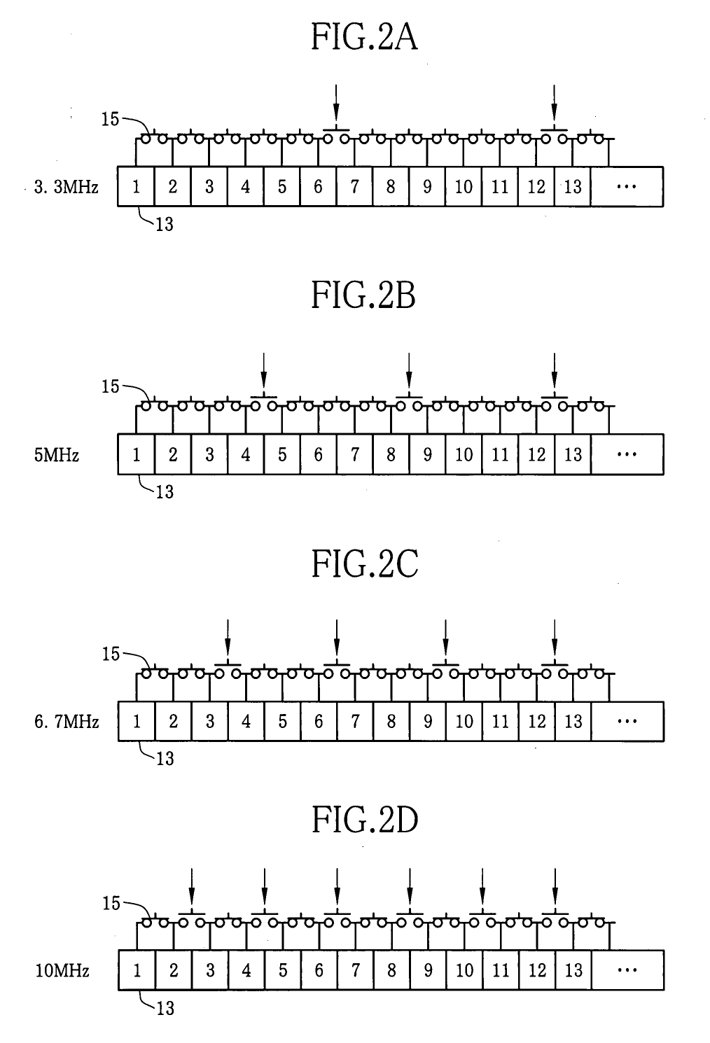

As shown in FIG. 2A, when the ultrasonic transducers 13 are driven at a frequency band of 3.3 MHz, every sixth short-circuit switch 15 is turned off (indicated by arrows in the figure), so that adjacent 6 (L=6) ultrasonic transducers 13 are simultaneously driven as a group. Similarly, as shown in FIGS. 2B to 2D, when the ultrasonic transducers 13 are driven at frequency bands of 5 MHz, 6.7 MHz, and 10 MHz, every forth, third, and second short-circuit switches 15 are turned off, so that adjacent 4 (L=4), 3 (L=3), and 2 (L=2) ultrasonic transducers 13 are simultaneously driven as a block respectively. That is, when the ultrasonic transducers 13 are driven at the frequency bands of 3.3 MHz, 5 MHz, 6.7 MHz, and 10 MHz, the width of each ultrasonic beam is 6 times, 4 times, 3 times and twice wider than that at 20 MHz respectively.

In FIG. 1, the wires 14 are connected to 48 (M=48) multiplexers (MUX) 16. To an mth (m=1 to 48) MUX 16, the wire 14 from the (48k+m)th (k=0 to 7) ultrasonic transducer 13 is connected. Eight wires 14 are connected per one MUX 16. The MUXs 16 selectively drive 48 out of 384 ultrasonic transducers 13. Every time the excitation pulse and the echo signal are transmitted to and received from the selected 48 ultrasonic transducers 13, the MUXs 16 selectively drive next 48 ultrasonic transducers 13, shifting one or several ultrasonic transducers 13 from the previously driven 48 ultrasonic transducers 13. Note that when the short-circuit switches 15 are turned on, 48×L ultrasonic transducers 13 are actually driven, but the L numbers of the ultrasonic transducers 13 are regarded as a single ultrasonic transducer.

The number L is a submultiple of the number M. In the above example, the M is 48, so it is preferable that L is one of 2, 3, 4, 6, 8, 12, 16, or 24. Thereby, it becomes possible to selectively drive 48×L ultrasonic transducers 13 only by controlling outputs of the MUXs 16 while the wires 14 connected to the MUXs 16 are fixed.

In the case each MUX 16 has 8 output terminals to which wires 14 are connected, the wire 14 from M (=48)×k+m (k=0 to 7)th ultrasonic transducer 13 is connected to the mth MUX 16 as described above. When the L ultrasonic transducers 13 are short-circuited, the mth MUX 16 selects the following ultrasonic transducers 13 to drive M×L ultrasonic transducers 13.

m mod L=1; m+L×k×M (≦N) (1)

m mod L=2; m+M+L×k×M (≦N) (2)

. . .

m mod L=0; m+(L−1)×M+L×k×M (≦N) (3)

m mod L=X on a left side of the semicolon indicates a number m which has a remainder X when m is divided by the number L. A mathematical expression on a right side of the semicolon indicates a number of the ultrasonic transducer 13 to be selected by the mth (the number m is indicated by the above m mod L) MUX 16. Note that m equals to the remainder X when m<L.

In other words, for instance, when M=48, and L=3, and m=1, 4, 7, . . . and 46, which corresponds to the above (1) when the remainder X=1, the mth MUX 16 selects mth, (m+144)th, and (m+288≦384)th ultrasonic transducers 13. When m=2, 5, 8, . . . and 47, which corresponds to above (2) when the remainder X=2, the mth MUX 16 selects (m+48)th, (m+192)th, and (m+336≦384)th ultrasonic transducers 13. When m=3, 6, 9, . . . and 48, which corresponds to the above (3) when the remainder X=0, the mth MUX 16 selects (m+96)th and (m+240≦384)th ultrasonic transducers 13.

A connecting wire 17 is connected to each MUX 16. The connecting wires 17 are shield lines, and bound together into a cable. At an end of the cable, a connecter (not shown) is provided. The connecter is inserted into a connecter (not shown) of the processor 11, and thus the ultrasonic probe 10 and the processor 11 are electrically connected.

The 48 connecting wires 17 are connected to the 48 transmitting/receiving circuits 18 respectively through the cable and the connecters. The transmitting/receiving circuits 18 switch the transmission of the ultrasonic waves and reception of the echo signals at predetermined time intervals.

A pulser 19, an amplifier 20, and a receiver 21 are connected to each of the transmitting/receiving circuits 18. The pulser 19 outputs an excitation pulse for generating the ultrasonic wave to the transmitting/receiving circuit 18. The amplifier 20 amplifies the echo signal output from the transmitting/receiving circuit 18. The receiver 21 receives the echo signal amplified by the amplifier 20.

The pulser 19 is connected to a timing controller 22. The receiver 21 is connected to a memory 23. Under the control of the CPU 24, the timing controller 22 outputs an excitation signal to the pulser 19 to generate the excitation pulse. The pulser 19 transmits the excitation pulse to the ultrasonic transducer 13 on the basis of this excitation signal. The memory 23 temporarily stores the echo signals received by the receiver 21.

A phase matching section 25 is connected to the memory 23. Under the control of the CPU 24, the phase matching section 25 provides a delay to each of the echo signals read from the memory 23 according to a time difference, and thereafter, adds the delayed echo signals.

The added echo signals are input in a display image processing section 26. The display image processing section 26 performs various image processing to the signals from the phase matching section 25, and converts the signals into NTSC signals. NTSC is a scan method for TV signals. A monitor 27 converts the NTSC signals into analog signals, and displays the analog signals as an ultrasonic image.

The operation unit 28 is connected to the CPU 24. The operation unit 28 is constituted of a keyboard, a mouse, a touch panel, or the like. The operation unit 28 is operated to change the frequency band of the ultrasonic transducers 13. The CPU 24 drives each section according to operation signals from the operation unit 28.

To obtain the ultrasonic image of a body part, first, the ultrasonic probe 10 is inserted into a body cavity of a living body from a forceps opening of an electronic endoscope. In the case the ultrasonic probe 10 is the ultrasonic endoscope, the ultrasonic probe 10 is inserted directly. The body part of the interest is searched by observing inside the body cavity with the electronic endoscope. In the case the ultrasonic probe 10 is the ultrasonic endoscope, the body cavity is observed with an imaging device disposed at a tip of the ultrasonic endoscope.

When the tip of the ultrasonic probe 10 reaches the body part of the interest, and a command to obtain the ultrasonic image is issued, under the control of the CPU 24, the MUX 16 selects the wire 14 connected to the ultrasonic transducer 13 to be driven. The excitation pulse is transmitted from the pulser 19 in response to the excitation signal from the timing controller 22.

The excitation pulse from the pulser 19 is transmitted to the ultrasonic transducer 13 through the transmitting/receiving circuit 18, the connecting wire 17, the MUX 16, and the wire 14. The ultrasonic transducer 13 is excited by the excitation pulse, and thus the ultrasonic wave is emitted toward the body part from the ultrasonic transducer 13.

After the ultrasonic wave is emitted, the echo signal from the body part is received by the ultrasonic transducer 13. The echo signal is transmitted through the wire 14, the MUX 16, the connecting wire 17, and the transmitting/receiving circuit 18, and amplified by the amplifier 20, and then received by the receiver 21. Every time the excitation pulse and the echo signal are transmitted and received, the MUX 16 selectively switches the ultrasonic transducers 13 to be driven next, and the above processing is performed in the same manner, and thus the body part is scanned with the ultrasonic waves.

The echo signal received by the receiver 21 is input and temporarily stored in the memory 23. Under the control of the CPU 24, the echo signals stored in the memory 23 are delayed according to the time difference, and added together in the phase matching section 25. The added echo signals are converted into the NTSC signals by the display image processing section 26. The NTSC signals are converted into the analog signals and displayed as the ultrasonic image on the monitor 27.

When the operation unit 28 is operated to change the frequency band of the ultrasonic transducers 13, the CPU 24 transmits the control signals to the short-circuit switches 15 in response to the operation signal input on the operation unit 28. Each of the short-circuit switch 15 is driven according to the transmitted control signal, and thus the ultrasonic beam is emitted with an appropriate beam width for the selected frequency band.

As described above, in accordance with the frequency band of the ultrasonic waves, the adjacent L numbers of the ultrasonic transducers 13 are short-circuited by the short-circuit switches 15, and the MUXs 16 selectively switch the 48 ultrasonic transducers 13 to be driven. As a result, the number of the connecting wires 17 is reduced from 384 to 48, and in this manner the advantage of the reduced number of wires 14, owing to the MUXs 16, can be fully utilized. In addition, the ultrasonic beam is emitted with the appropriate beam width in accordance with the frequency band. Accordingly, it becomes unnecessary to reduce the interval between the ultrasonic transducers 13 to increase the number of the ultrasonic transducers 13 for the high frequency bands, or increase the intervals between the ultrasonic transducers 13 to reduce the number of the ultrasonic transducers 13 for the low frequency bands.

As described in the above embodiment, it is especially effective when the present invention is applied to the intracavity probes and the ultrasonic endoscopes which require wide frequency bands and very small diameters. Further, since the ultrasonic transducers 13 having the frequency band of approximately 3 MHz to 20 MHz are used, it becomes possible to use a single probe throughout the diagnosis and to avoid inserting and removing plural probes having different frequency bands.

In the above embodiment, the radial scan type probe is described. The radial scan type probe carries out ultrasonographic scanning by simultaneously driving 48 out of 384 ultrasonic transducers as one block and sequentially switching the blocks to be driven. However, it is also possible to apply the present invention to a so-called sector scan type probe which sequentially drives the ultrasonic transducers 13 at a predetermined delay. In this case, the delay is controlled by the timing controller 22.

In the sector scan type, grating lobes occur easily when the interval between the ultrasonic transducers is broad. By the application of the present invention, the interval is reduced and the occurrence of the grating lobes is also reduced.

In the above embodiment, the number L of the ultrasonic transducers 13 short-circuited by the short-circuit switches 15 is changed in accordance with the frequency band selected on the operation unit 28. Instead, or in addition, it is also possible to use the operation unit capable of inputting the focal distance of the ultrasonic beam. The number L can be changed in accordance with the focal distance. In this case, the number L increases as the focal distance increases.

Values such as the number of the ultrasonic transducers in the ultrasonic transducer array, and the number of the ultrasonic transducers to be driven simultaneously are mere examples and do not limit the scope of the present invention.

As described so far, the present invention is not to be limited to the above embodiments, and all matter contained herein is illustrative and does not limit the scope of the present invention. Thus, obvious modifications may be made within the spirit and scope of the appended claims.

What is claimed is:

1. An ultrasonic probe comprising:
N numbers of ultrasonic transducers disposed on a tip of said ultrasonic probe;
a multiplexer for selectively switching M numbers of said ultrasonic transducers to be driven out of said N numbers of said ultrasonic transducers; and
a short-circuit device for short-circuiting adjacent L numbers of said ultrasonic transducers out of said N numbers of said ultrasonic transducers to simultaneously drive said L numbers of said ultrasonic transducers.

2. An ultrasonic probe according to claim 1, wherein said number L is changed in accordance with a frequency band of said ultrasonic waves.

3. An ultrasonic probe according to claim 1, wherein said number L is changed in accordance with a focal distance of said ultrasonic waves.

4. An ultrasonic probe according to claim 1, wherein said number L is a submultiple of said number M.

5. An ultrasonic probe according to claim 1, wherein said ultrasonic probe is inserted in a body cavity for intracavity diagnosis.

6. An ultrasonic probe according to claim 1, wherein said ultrasonic probe is an ultrasonic endoscope having an image sensor for capturing an optical image of a target region.

7. An ultrasonic probe according to claim 1, wherein said ultrasonic transducers transmit and receive said ultrasonic waves in a frequency band of approximately 3 to 20 MHz.

8. An ultrasonic probe according to claim 1, wherein said M numbers of said ultrasonic transducers are sequentially driven at a predetermined delay time.

9. An ultrasonic diagnosing apparatus constituted of an ultrasonic probe having N numbers of ultrasonic transducers on a tip thereof, and a processor for generating an ultrasonic image from an echo signal from a target region received by said ultrasonic transducers, said ultrasonic diagnosing apparatus comprising:
said ultrasonic probe including:
a multiplexer for selectively switching M numbers of said ultrasonic transducers to be driven out of said N numbers of said ultrasonic transducers; and
a short-circuit device for short-circuiting adjacent L numbers of said ultrasonic transducers out of said N numbers of said ultrasonic transducers to simultaneously drive said L numbers of said ultrasonic transducers;
said processor including:
a control unit for controlling an operation of said short-circuit device.

10. An ultrasonic diagnosing apparatus according to claim 9, further including:
a first operation device for changing a frequency band of said ultrasonic waves, wherein said control unit changes said number L in accordance with an operation of said first operation device.

11. An ultrasonic diagnosing apparatus according to claim 9, further comprising:
a second operation device for changing a focal distance of said ultrasonic waves, wherein said control unit changes said number L in accordance with an operation of said second operation device.

12. An ultrasonic diagnosing apparatus according to claim 9, wherein said number L is a submultiple of said number M.

13. An ultrasonic diagnosing apparatus according to claim 9, wherein said ultrasonic probe is inserted into a body cavity for intracavity diagnosis.

14. An ultrasonic diagnosing apparatus according to claim 9, wherein said ultrasonic probe is an ultrasonic endoscope having an image sensor for capturing an optical image of a target region.

15. An ultrasonic diagnosing apparatus according to claim 9, wherein said ultrasonic transducers transmit and receive said ultrasonic waves in a frequency band of approximately 3 to 20 MHz.

16. An ultrasonic diagnosing apparatus according to claim 9, further including a driving device for sequentially driving said M numbers of said ultrasonic transducers at a predetermined delay time.
